(19) United States
(12) Patent Application Publication
(10) Pub. No.: US 2012/0005128 A1
(43) Pub. Date: Jan. 5, 2012

(54) METHODS FOR REDUCING THE RISK OF AN ADVERSE DRONEDARONE / CALCIUM CHANNEL BLOCKERS INTERACTION IN A PATIENT SUFFERING FROM ATRIAL FIBRILATION

(75) Inventors: Christine GAUD, Montpellier (FR); Davide RADZIK, Paris (FR)

(73) Assignee: sanofi, Paris (FR)

(21) Appl. No.: 13/172,336

(22) Filed: Jun. 29, 2011

Related U.S. Application Data
(60) Provisional application No. 61/359,608, filed on Jun. 29, 2010.

(51) Int. Cl. A61K 31/343; A61P 9/06; G06Q 90/00; A61P 9/00

(52) U.S. Cl. 705/500; 514/469

(57) ABSTRACT

The disclosure relates to a method for managing the risk of dronedarone/calcium channel blockers interaction by using dronedarone or pharmaceutically acceptable salts thereof in patients with paroxysmal or persistent atrial fibrillation (AF) or atrial flutter (AFL), with a recent episode of AF/AFL and associated cardiovascular risk factors, who are in sinus rhythm or who will be cardioverted to reduce the risk of cardiovascular hospitalization, said patients also expecting to receive a calcium channel blockers treatment, by performing the following steps:
a—initiate calcium channel blockers treatment at a low dose;
b—performing a electrocardiogram (ECG) verification of good tolerability;
c—increase of calcium channel blockers dose only if results in step b) are satisfying.

The present invention relates to the use of dronedarone or pharmaceutically acceptable salts thereof, for the preparation of a medicament for use in the prevention of atrial fibrillation, and reducing the risk of an adverse drug interaction with calcium channel blockers, verapamil particularly.

The instant invention relates to a method of providing dronedarone or pharmaceutically acceptable salts thereof.

The instant invention also relates to a method of managing the risk of a calcium channel blocker such as verapamil/dronedarone interaction.

The instant invention also relates to a method of promoting the use of dronedarone or pharmaceutically acceptable salts or esters thereof.

The instant invention also relates to an article of manufacture and a package comprising dronedarone or pharmaceutically acceptable salts or esters thereof.

2-n-Butyl-3-[4-(3-di-n-butylaminopropoxy)benzoyl]-5-methylsulphonamidobenzofuran, or dronedarone, and pharmaceutically acceptable salts thereof are described in European Patent EP 0 471 609 B1.

Dronedarone blocks potassium, sodium and calcium channels and also has anti-adrenergic properties. Dronedarone is an antiarrhythmic that is effective in maintaining sinus rhythm in patients presenting atrial fibrillation or atrial flutter.

The applicant has clinically proven that dronedarone significantly reduces cardiovascular hospitalizations and/or mortality in patients having a history of atrial fibrillation (AF) or of atrial flutter (AFL) in a safe and effective way. Dronedarone is indicated to reduce the risk of cardiovascular hospitalization in patients with paroxysmal or persistent AF or AFL, with a recent episode of AF/AFL and associated cardiovascular risk factors (i.e., age >70, hypertension, diabetes, prior cerebrovascular accident, left atrial diameter ≧50 mm or left ventricular ejection fraction [LVEF] <40%), who are in sinus rhythm or who will be cardioverted.

Calcium channel blockers (CCB), commonly prescribed drugs in cardiac patients, are mainly metabolized by CYP3A4. Dronedarone has a potential to inhibit CYP3A4. Interactions due to inhibition of CCB metabolism by CYP3A4 inhibitors have been reported.

Verapamil which is a calcium channel blocker belongs to class IV of antiarrhythmics and decreases impulse conduction through the atrioventricular node. CCBs and in particular verapamil are frequently co-prescribed as they are used for the treatment of hypertension, supraventricular arrhythmias, and angina pectoris. The coadministration of dronedarone and verapamil may result in an interaction ie in potentiation of the depressant effect on conduction and may lead to bradycardia, hypotension or heart failure if not carefully managed. Based on the likelihood of the co-prescription of these 2 drugs and the likelihood of clinically significant interactions, an interaction study was performed.

During interaction study with dronedarone, it has been observed that administration of this active principle together with CCB such as verapamil is associated with an increase of steady state CCB exposures, which can lead to potentiation of the depressant effect on conduction.

The Applicant has now found the regimen to administrate dronedarone to patients in a safe and effective way, those patients expecting to receive calcium channel blockers such as verapamil. Consequently, they found methods for managing the risk related to calcium channel blockers such as verapamil/dronedarone interaction.

The methods according to the invention enable to decrease the risk of such interaction, when dronedarone or pharmaceutically acceptable salts or esters thereof is administered for treating patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF] <40%), who are in sinus rhythm or who will be cardioverted.

The present invention relates to the use of dronedarone or pharmaceutically acceptable salts thereof in patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF] <40%), who are in sinus rhythm or who will be cardioverted to reduce the risk of cardiovascular hospitalization, said patients also expecting to receive a calcium channel blockers treatment, by performing the following steps
a—initiate calcium channel blockers treatment at a low dose;
b—performing electrocardiogram (ECG) verification of good tolerability;
c—increase calcium channel blockers dose only if results in step b) are satisfying.

The present invention also relates to a method for managing the risk of dronedarone/calcium channel blockers interaction by using dronedarone or pharmaceutically acceptable salts thereof in patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF] <40%), who are in sinus rhythm or who will be cardioverted to reduce the risk of cardiovascular hospitalization, said patients also expecting to receive a calcium channel blockers treatment, by performing the following steps:
a—initiate calcium channel blockers treatment at a low dose;
b—performing ECG verification of good tolerability;
c—increase of calcium channel blockers dose only if results in step b) are satisfying.

In the instant invention, calcium channel blockers also called calcium antagonists are those with depressant effects on the sinus and AV nodes, i.e., with heart rate lowering effect such as verapamil, diltiazem or bepridil.

In some embodiments, dronedarone is administered 400 mg twice daily with meals.

In some embodiments a pharmaceutically acceptable salt of dronedarone is hydrochloride.

In step a), the term “low dose of calcium channel blockers” as used herein means a fraction of the recommended dose. In any case, this low dose is patient dependent and may be defined taking into account individual patients' characteristics.

For example, a low dose may be
for verapamil, less than 120 mg BID e.g. as 40 mg BID;
for diltiazem, less than 60 mg TID, e.g. as a fraction of 60 mg TID,
for bepridil, less than 200 mg OD e.g. as 100 mg OD.

In step b), “ECG verification of good tolerability” and “ECG assessment” has the same meaning, i.e. assessments have to be made regarding for example heart rate, PR- and QTc-interval duration. For example heart rate should be higher than 50 bpm, QTc-interval should be shorter than 500 ms.

In step c), increase or uptitration of calcium channel blockers will be a fraction of the recommended dose higher than the low dose as defined above up to a maximum dose of:
240 mg BID for verapamil;
360 mg daily dose for diltiazem;
300 mg OD for bepridil.

The recommended dose is the dose defined in the labelling of the calcium channel blockers.

The maximum dose is defined according to the labelling of the calcium channel blockers.

The term “cardiovascular hospitalization” means a hospitalization which is caused by at least one of the following pathologies (Hohnloser et al., Journal of cardiovascular electrophysiology, January 2008, vol. 19, No. 1, pages 69-73):
relating to atherosclerosis,
myocardial infarction or unstable angina pectoris,
stable angina pectoris or atypical thoracic pain,
syncope,
transient ischemic event or cerebral stroke (except intracranial haemorrhage),
atrial fibrillation and other supraventricular rhythm disorders,
non-fatal cardiac arrest,
ventricular arrhythmia,
cardiovascular surgery, except heart transplant,
heart transplant,
implantation of a cardiac stimulator (pacemaker), of an implantable defibrillator (“ICD”) or of another cardiac device,
percutaneous coronary, cerebrovascular or peripheral intervention,
variations in arterial pressure (hypotension, hypertension, except syncope),
cardiovascular infection,
major bleeding/haemorrhage (requiring two or more blood cell pellets or any intracranial haemorrhage),
pulmonary embolism or deep vein thrombosis,
worsening of congestive heart failure including acute pulmonary oedema or dyspnoea from cardiac causes.

Consequently, the prevention of cardiovascular hospitalization may be understood as the prevention of cardiovascular hospitalization for at least one of the above mentioned pathologies.

The present invention also relates to a method of providing dronedarone or pharmaceutically acceptable salts or esters, wherein said dronedarone or pharmaceutically acceptable salts or esters thereof is provided along with information indicating that it is useful for treating patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF] <40%), who are in sinus rhythm or who will be cardioverted to reduce the risk of cardiovascular hospitalization, and in case said patients also expecting to receive a calcium channel blockers treatment, the following steps should be observed:
a—initiate calcium channel blockers treatment at a low dose;
b—performing ECG verification of good tolerability;
c—increase of calcium channel blockers dose only if results in step b) are satisfying.

In some embodiments a pharmaceutically acceptable salt of dronedarone is hydrochloride. In some embodiments, the information comprises printed matter that advises that dronedarone or pharmaceutically acceptable salts or esters thereof is useful for treating patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF] <40%), who are in sinus rhythm or who will be cardioverted to reduce the risk of cardiovascular hospitalization, and in case said patients also expecting to receive a calcium channel blockers treatment, the following steps should be observed:
a—initiate calcium channel blockers treatment at a low dose;
b—performing ECG verification of good tolerability;
c—increase of calcium channel blockers dose only if results in step b) are satisfying.

The present invention also concerns a method of promoting the use of dronedarone or pharmaceutically acceptable salts or esters thereof, the method comprising the step of conveying to a recipient at least one message comprising dronedarone or pharmaceutically acceptable salts or esters thereof is useful for treating patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF] <40%), who are in sinus rhythm or who will be cardioverted to reduce the risk of cardiovascular hospitalization, and in case said patients also expecting to receive a calcium channel blockers treatment, the following steps should be observed:
a. initiate calcium channel blockers treatment at a low dose;
b. performing ECG verification of good tolerability;
c. increase of calcium channel blockers dose only if results in step b) are satisfying.

In some embodiments a pharmaceutically acceptable salt of dronedarone is hydrochloride. The instant invention also concerns an article of manufacture comprising
a) a packaging material;
b) dronedarone or pharmaceutically acceptable salts or esters thereof or, and
c) a label or package insert contained within the packaging material indicating that:

i) dronedarone or pharmaceutically acceptable salts or esters thereof is indicated in patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF] <40%), who are in sinus rhythm or who will be cardioverted to reduce the risk of cardiovascular hospitalization, and

ii) in case said patients also expecting to receive a calcium channel blockers treatment, the following steps should be observed:
a. initiate calcium channel blockers treatment at a low dose;
b. performing ECG verification of good tolerability;
c. increase of calcium channel blockers dose only if results in step b) are satisfying.

In some embodiments a pharmaceutically acceptable salt of dronedarone is hydrochloride.

The invention also concerns a package comprising dronedarone or pharmaceutically acceptable salts or esters thereof and a label, said label comprising a printed statement which informs a prospective user that:

i) dronedarone or pharmaceutically acceptable salts or esters thereof is indicated in patients with paroxysmal or persistent atrial fibrillation (AF) or atrial flutter (AFL), with a recent episode of AF/AFL and associated cardiovascular risk factors (i.e., age >70, hypertension, diabetes, prior cerebrovascular accident, left atrial diameter ≧50 mm or left ventricular ejection fraction [LVEF] <40%), who are in sinus rhythm or who will be cardioverted to reduce the risk of cardiovascular hospitalization, and

ii) in case said patients also expecting to receive a calcium channel blockers treatment, the following steps should be observed:
a. initiate calcium channel blockers treatment at a low dose;
b. performing ECG verification of good tolerability;
c. increase of calcium channel blockers dose only if results in step b) are satisfying.

In some embodiments a pharmaceutically acceptable salt of dronedarone is hydrochloride.

The invention also relates to a method for administering a combination of calcium channel blockers and dronedarone or a pharmaceutically acceptable salt thereof to a patient in need thereof, wherein said method comprises the following steps:
a. initiate calcium channel blockers treatment at a low dose;
b. performing ECG verification of good tolerability;
c. increase of calcium channel blockers dose only if results in step b) are satisfying.

In some embodiments a pharmaceutically acceptable salt of dronedarone is hydrochloride

The term “pharmaceutically acceptable salts” as used herein means that the salts of the compounds of the present invention can be used in medicinal preparations. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, hydrobromic acid, sulfuric acid, methanesulfonic acid, 2-hydroxyethanesulfonic acid, p-toluenesulfonic acid, fumaric acid, maleic acid, hydroxymaleic acid, malic acid, ascorbic acid, succinic acid, glutaric acid, acetic acid, salicylic acid, cinnamic acid, 2-phenoxybenzoic acid, hydroxybenzoic acid, phenylacetic acid, benzoic acid, oxalic acid, citric acid, tartaric acid, glycolic acid, lactic acid, pyruvic acid, malonic acid, carbonic acid or phosphoric acid. The acid metal salts such as sodium monohydrogen orthophosphate and potassium hydrogen sulfate can also be formed. Also, the salts so formed may present either as mono- or di-acid salts and can exist substantially anhydrous or can be hydrated. Furthermore, where the compounds of the invention carry an acidic moiety, suitable pharmaceutically acceptable salts thereof may include alkali metal salts, e.g. sodium or potassium salts; alkaline earth metal salts, e.g. calcium or magnesium salts, and salts formed with suitable organic ligands, e.g. quaternary ammonium salts.

It will also be specified that the expression “patients having a history of atrial fibrillation or atrial flutter”, “patients with a history of or a current atrial fibrillation or flutter” or “patients with a recent history of or a current atrial fibrillation or flutter” or “patients with paroxysmal or persistent atrial fibrillation or flutter” or “patients with a history of, or a current paroxysmal or persistent atrial fibrillation or flutter” or “patients with a recent history of, or a current paroxysmal or persistent atrial fibrillation or flutter” or “patients with paroxysmal or intermittent atrial fibrillation or atrial flutter and a recent episode of atrial fibrillation or atrial flutter, who are in sinus rhythm or who will be cardioverted” or “patients with paroxysmal or persistent atrial fibrillation or atrial flutter and a recent episode of atrial fibrillation or atrial flutter, who are in sinus rhythm or who will be cardioverted” means a patient who, in the past, has presented one or more episodes of atrial fibrillation or flutter and/or who is suffering from atrial fibrillation or atrial flutter at the time the dronedarone or a pharmaceutically acceptable salt thereof is used. More particularly, this expression means patients with documentation of having been in both atrial fibrillation or flutter and sinus rhythm within the last 6 months preceding the start of treatment. Patients could be either in sinus rhythm, or in atrial fibrillation or flutter at the time the dronedarone or a pharmaceutically acceptable salt thereof is initiated.

The instant invention is further illustrated by the clinical data below.

### EXAMPLE 1

This was a randomized, open-label, 3-period, three-treatment crossover study with 14-day washouts, in 21 healthy male subjects. Subjects received dronedarone 400 mg BID alone, verapamil (VP) 240 mg OD alone, dronedarone and VP co-administration at same regimens described above for 14 days. All treatments were administered in fed conditions. Blood samples were collected at trough during the repeated administration, then over 12 h after the last dronedarone dose for dronedarone/SR35021 for dronedarone/SR35021 analysis, and up to 24 h after the last verapamil dose for VP/norverapamil (NVP) analysis.

Plasma dronedarone/SR35021 concentrations were determined using a validated LC-MS/MS method, verapamil and norverapamil plasma concentrations were assessed using a validated LC-UV detection method.

A noncompartmental PK analysis was performed using validated in-house software running on an Oracle (7.1.5) database installed on VAX-VMS. The following parameters were assessed:
Cₘₐₓ (maximum observed concentration)

tₘₐₓ (time to maximum observed concentration)
AUC₀₋₁₂ (area under the plasma concentration time curve over 12 h for dronedarone and SR35021)
AUC₀₋₂₄ (area under the plasma concentration time curve over 24 h for verapamil and norverapamil).

HR, PR-, QRS-, QT-intervals and QTcB/F were determined from manual reading of 12-lead ECGs performed at specified timepoints up to 24 h postdose on Day 14.

Statistical Analysis
Cₘₐₓ and AUC₀₋₂₄ values for verapamil and norverapamil, and Cₘₐₓ and AUC₀₋₁₂ values for dronedarone and SR35021, were log transformed; tmax values were rank transformed. These transformed PK parameters were analyzed with a linear mixed-effect model with fixed terms for sequence, period, and treatment, and random term for subject within sequence.
Estimates and 90% confidence intervals (CIs) for the difference in means between the treatments (verapamil plus dronedarone versus verapamil alone; dronedarone plus verapamil versus dronedarone alone) were computed within the mixed-model framework, and then converted to ratios by antilog transformation. Otherwise, the magnitude of the effect of dronedarone on verapamil/norverapamil PK or the effect of verapamil on dronedarone/SR35021 PK was based on the estimated ratios and 90% CIs.
Mean changes (Eavr) from baseline for PR- and Bazett/Fridericia-corrected QT (QTcB/F) intervals were analyzed using a linear mixed-effect model. Results of pairwise comparisons between co-administration versus single-agent treatment were presented as mean differences with 95% CIs.

Results

Subjects
21 healthy white male subjects were recruited. Mean (±SD) age was 30.6±5.3 years (range, 22-40) and mean body mass index (±SD) was 23.64±1.93 kg/m2 (range, 8.8-27.2).
3 subjects discontinued the study: 1 subject was withdrawn because of an adverse event (mild maculopapular rash); 2 subjects did not receive treatment (1 withdrew consent and the other tested positive for cannabis/cocaine).

TABLE 1
Mean (CV %) PK parameters of VP/NVP and dronedarone/SR35021 and their ratio
of geometric means (90% confidence interval, CI) are shown in the table 1.

| | | Dronedarone | VP | | Ratio |
|---|---|---|---|---|---|
| Analyte | Parameter | alone | alone | VP + Dronedarone | (90% CI) |
| VP | Cₘₐₓ | | 132 (59) | 175 (42) | 1.40 |
| | (ng/mL) | | | | [1.13, 1.73] |
| | AUC₀₋₂₄ | | 1670 (46) | 2130 (34) | 1.30 |
| | (ng · h/mL) | | | | [1.14, 1.48] |
| NVP | Cₘₐₓ | | 129 (45) | 169 (51) | 1.31 |
| | (ng/mL) | | | | [1.08, 1.59] |
| | AUC₀₋₂₄ | | 2150 (37) | 2740 (27) | 1.29 |
| | (ng · h/mL) | | | | [1.15, 1.44] |
| Dronedarone | Cₘₐₓ | 135 (32) | | 188 (30) | 1.42 |
| | (ng/mL) | | | | [1.31, 1.53] |
| | AUC₀₋₁₂ | 895 (34) | | 1310 (35) | 1.48 |
| | (ng · h/mL) | | | | [1.38, 1.58] |
| SR35021 | Cₘₐₓ | 98.0 (33) | | 88.2 (19) | 0.93 |
| | (ng/mL) | | | | [0.83, 1.04] |
| | AUC₀₋₁₂ | 781 (27) | | 742 (19) | 0.98 |
| | (ng · h/mL) | | | | [0.92, 1.04] |

PK Assessments

Verapamil
Plasma concentrations of verapamil on Day 14 were higher after co-administration with dronedarone than after administration of verapamil alone. Similarly, plasma concentrations of norverapamil on Day 14 were higher after co-administration with dronedarone than after administration of verapamil alone.
Mean plasma PK parameters for verapamil and norverapamil on Day 14, after 14 days of administration of verapamil plus dronedarone or verapamil alone, and mean ratio estimates with 90% Cls, are shown in Table 1. Co-administration of dronedarone with verapamil increased verapamil and norverapamil exposures 1.3-fold (90% Cls: for verapamil 1.14-1.48; for norverapamil 1.15-1.44).

Dronedarone
Plasma concentrations of dronedarone on Day 14 were higher after co-administration with verapamil than after administration of dronedarone alone. Plasma concentrations of SR35021 on Day 14 were unaffected by co-administration of verapamil.
Mean plasma PK parameters for dronedarone and SR35021 on Day 14, after 14 days of administration of dronedarone plus verapamil or dronedarone alone, and mean ratio estimates with 90% Cls, are shown in Table 1. Co-administration of verapamil with dronedarone increased dronedarone exposure 1.5-fold (90% CI, 1.38-1.58), but SR35021 exposure was unaffected.

Electrocardiography

Co-administration of dronedarone and verapamil resulted in greater increases in the PR and Fridericia-corrected QT (QTcF) intervals than administration of each drug alone (Table 2). Mean differences in PR and QTcF intervals between co-administration and dronedarone alone were +10.2 ms and +11.9 ms, respectively. Mean differences in PR and QTcF intervals between co-administration and verapamil alone were +15.8 ms and +31.9 ms, respectively.

TABLE 2
Mean changes (Eavr) in ECG parameters from baseline (Day 1, −0.5 h) on Day 14.

| | | Mean^{b} | | | |
|---|---|---|---|---|---|
| | Treatment | Difference | | | |
| Change from | contrast (co- | (Co- | Co- | Reference^{d} | |
| baseline in | administration | administration- | administration | mean^{c} | |
| ECG | versus | reference) | mean^{c} change | change | |
| parameter | reference)^{a} | (ms)^{a} | (ms) | (ms) | 95% CI |
| Change PR | co- | 10.2 | 19.9 | 9.7 | 2.6-17.8 |
| (ms) | administration | | | | |
| | versus | | | | |
| | dronedarone | | | | |
| | co- | 15.8 | 19.9 | 4.2 | 8.2-23.4 |
| | administration | | | | |
| | versus | | | | |
| | verapamil | | | | |
| Change | co- | 11.9 | 33.9 | 22.1 | 1.5-22.3 |
| QTcF (ms) | administration | | | | |
| | versus | | | | |
| | dronedarone | | | | |
| | co- | 31.9 | 33.9 | 2.1 | 21.4-42.3 |
| | administration | | | | |
| | versus | | | | |
| | verapamil | | | | |

^{a}Co-administration is dronedarone + verapamil; reference is either verapamil or dronedarone.
^{b}Mean difference = least square means (co-administration) minus least square means (reference).
^{c}Mean is least square means.
^{d}Sample size for reference group (n = 16 for verapamil alone and n = 16 for dronedarone alone for all end points).
PR = PR interval;
QTcF = Fridericia corrected QT interval.

Safety
No deaths or serious adverse events were reported.

Conclusions

Dronedarone increased verapamil/norverapamil steady state exposure by 1.3-fold. Verapamil increased dronedarone steady state exposure by 1.5-fold but did not modify the exposure of the active metabolite. Co-administration of dronedarone and VP increased PR duration and QTcF greater than each drug alone with no cardiovascular adverse events or arrhythmia. It is recommended that the use of verapamil together with dronedarone be done with caution. Due to alterations in ECG parameters, verapamil should be initiated at a low dose in patients receiving dronedarone, and up-titration of verapamil should be done only after ECG assessment

Calcium channel blockers have been used in ATHENA clinical study and at the same rate in the dronedarone group and in the placebo group. About 13 to 20% of the patients had received one of these compounds during the study. Table 5 shows the numbers and percentages of patients using calcium antagonists with heart rate lowering effects at the inclusion in the study, whereas table 6 shows the numbers and percentages of patients who received calcium antagonists with heart rate lowering effects as concomitant medications during the study.

TABLE 5
Number (%) baseline selected medications - All randomized patients

| | | Dronedarone | |
|---|---|---|---|
| | Placebo | 400 mg BID | Total |
| | (N = 2327) | (N = 2301) | (N = 4628) |
| Calcium antagonists with | 307 (13.2%) | 331 (14.4%) | 638 (13.8%) |
| heart rate lowering effects | | | |

TABLE 6
Number (%) of patients who received concomitant medications - All
randomized patients

| | | Dronedarone |
|---|---|---|
| | Placebo | 400 mg BID |
| | (N = 2327) | (N = 2301) |
| Calcium antagonists with heart rate lowering | 490 (21.1%) | 459 (19.9%) |
| effects | | |

In the ATHENA trial, the concomitant administration of calcium antagonists and dronedarone was done according to the above mentioned instructions, i.e. start with low doses of the calcium antagonist and then increase the dose after ECG verification of good tolerability, if necessary and based on individual patient characteristics.

In the ATHENA trial, in the patients using both dronedarone and calcium antagonists, there was no increase of the risk of hypotension, bradycardia and heart failure. In these patients, dronedarone has been shown to be effective for the prevention of mortality and morbidity (hospitalization) notably for the prevention of hospitalization for congestive heart failure.

The relative risk (RR) was estimated using Cox's proportional-effect regression model.

The relative risk (RR) is the ratio of the rates of occurrence of a hospitalization or of a death among the patients on dronedarone, relative to the patients on placebo.

The percentage reduction x of a given event (hospitalization, death, cardiovascular death, etc.) is calculated in the following way:

x=1−relative risk.

Results Relating to the Prevention of Cardiovascular Hospitalization or Death

From the 4628 patients included in the trial, 2301 were part of the group treated with dronedarone hydrochloride.

307 patients with calcium channel blockers baseline medication were part of the placebo group and 331 patients with calcium channel blockers baseline medication were part of the group treated with dronedarone hydrochloride.

122 events were reported in the placebo group versus 90 in the group treated with dronedarone hydrochloride.

Calculated relative risk was equal to 0.628, i.e. a decrease of cardiovascular hospitalization or death of 37% in patients with calcium channel blockers baseline medication.

Results Relating to the Prevention of Death

From the 4628 patients included in the trial, 2301 were part of the group treated with dronedarone hydrochloride.

307 patients with calcium channel blockers baseline medication were part of the placebo group and 331 patients with calcium channel blockers baseline medication were part of the group treated with dronedarone hydrochloride.

122 events were reported in the placebo group versus 90 in the group treated with dronedarone hydrochloride.

Calculated relative risk was equal to 0.628, i.e. a decrease of death of 37% in patients with calcium channel blockers baseline medication.

Results Relating to the Prevention of Hospitalization for Congestive Heart Failure (CHF)

From the 4628 patients included in the trial, 2301 were part of the group treated with dronedarone hydrochloride.

307 patients with calcium channel blockers baseline medication were part of the placebo group and 331 patients with calcium channel blockers baseline medication were part of the group treated with dronedarone hydrochloride.

23 events were reported in the placebo group versus 14 in the group treated with dronedarone hydrochloride.

Calculated relative risk was equal to 0.556, i.e. a decrease of CHF of 44% in patients with calcium channel blockers baseline medication.

What is claimed is:

1. A method for managing the risk of dronedarone/calcium channel blockers interaction by administering dronedarone or pharmaceutically acceptable salt thereof in patients with paroxysmal or persistent atrial fibrillation (AF) or atrial flutter (AFL), with a recent episode of AF/AFL and associated cardiovascular risk factors, who are in sinus rhythm or who will be cardioverted to reduce the risk of cardiovascular hospitalization, said patients also expecting to receive a calcium channel blockers treatment, by performing the following steps:
a—initiating calcium channel blockers treatment at a low dose;
b—performing a electrocardiogram (ECG) verification of good tolerability; and
c—increasing calcium channel blockers dose only if results in step b) are satisfying.

2. The method according to claim 1, wherein 400 mg of dronedarone is administered twice daily with meals.

3. The method according to claim 1, wherein the pharmaceutically acceptable salt of dronedarone is hydrochloride.

4. The method according to claim 1, wherein in step a), a low dose of calcium channel blockers is as a fraction of the recommended dose.

5. The method according to claim 1, wherein in step b), the calcium channel blocker dose is increased up to a fraction of the recommended dose higher than the low dose.

6. A method of providing dronedarone or a pharmaceutically acceptable salt or ester, wherein said dronedarone or pharmaceutically acceptable salt or ester thereof is provided along with information indicating that it is useful for treating patients with paroxysmal or persistent atrial fibrillation (AF) or atrial flutter (AFL), with a recent episode of AF/AFL and associated cardiovascular risk factors, who are in sinus rhythm or who will be cardioverted to reduce the risk of cardiovascular hospitalization, and in case said patients also receive a calcium channel blockers treatment, the following steps should be observed:
a—initiate calcium channel blockers treatment at a low dose;
b—performing ECG verification of good tolerability;
c—increase of calcium channel blockers dose only if results in step b) are satisfying.

7. The method according to claim 6, wherein the pharmaceutically acceptable salt of dronedarone is hydrochloride.

8. The method according to claim 6, wherein the information comprises printed matter that advises that dronedarone or pharmaceutically acceptable salt or ester thereof is useful for treating patients with paroxysmal or persistent atrial fibrillation (AF) or atrial flutter (AFL), with a recent episode of AF/AFL and associated cardiovascular risk factors, who are in sinus rhythm or who will be cardioverted to reduce the risk of cardiovascular hospitalization, and in case said patients also receive a calcium channel blockers treatment, the following steps should be observed:
a—initiate calcium channel blockers treatment at a low dose;
b—performing ECG verification of good tolerability;
c—increase of calcium channel blockers dose only if results in step b) are satisfying.

9. A method of promoting the use of dronedarone or pharmaceutically acceptable salt or ester thereof, the method comprising the step of conveying to a recipient at least one message comprising dronedarone or pharmaceutically acceptable salts or esters thereof is useful for treating patients with paroxysmal or persistent atrial fibrillation (AF) or atrial flutter (AFL), with a recent episode of AF/AFL and associated cardiovascular risk factors, who are in sinus rhythm or who will be cardioverted to reduce the risk of cardiovascular hospitalization, and in case said patients also receive a calcium channel blockers treatment, the following steps should be observed:
a—initiate calcium channel blockers treatment at a low dose;
b—performing ECG verification of good tolerability;
c—increase of calcium channel blockers dose only if results in step b) are satisfying.

10. The method according to claim 9, wherein the pharmaceutically acceptable salt of dronedarone is hydrochloride.

11. An article of manufacture comprising
a) a packaging material;
b) dronedarone or pharmaceutically acceptable salt or ester thereof or, and
c) a label or package insert contained within the packaging material indicating that:
i) dronedarone or pharmaceutically acceptable salt or ester thereof is indicated in patients with paroxysmal or persistent atrial fibrillation (AF) or atrial flutter (AFL), with a recent episode of AF/AFL and associated cardiovascular risk factors, who are in sinus rhythm or who will be cardioverted to reduce the risk of cardiovascular hospitalization, and
ii) in case said patients also expect to receive a calcium channel blockers treatment, the following steps should be observed:
a—initiate calcium channel blockers treatment at a low dose;
b—performing ECG verification of good tolerability;
c—increase of calcium channel blockers dose only if results in step b) are satisfying.

12. The article according to claim 11, wherein the pharmaceutically acceptable salt of dronedarone is hydrochloride.

13. A package comprising dronedarone or pharmaceutically acceptable salt or ester thereof and a label, said label comprising a printed statement which informs a prospective user that:
i) dronedarone or pharmaceutically acceptable salt or ester thereof is indicated in patients with paroxysmal or persistent atrial fibrillation (AF) or atrial flutter (AFL), with a recent episode of AF/AFL and associated cardiovascular risk factors, who are in sinus rhythm or who will be cardioverted to reduce the risk of cardiovascular hospitalization, and
ii) in case said patients also receive a calcium channel blockers treatment, the following steps should be observed:
a—initiate calcium channel blockers treatment at a low dose;
b—performing ECG verification of good tolerability;
c—increase of calcium channel blockers dose only if results in step b) are satisfying.

14. The package according to claim 13, wherein the pharmaceutically acceptable salt of dronedarone is hydrochloride.

15. A method for administering a combination of verapamil and dronedarone or a pharmaceutically acceptable salt thereof to a patient in need thereof, wherein said method comprises the following steps:
a—initiating calcium channel blockers treatment at a low dose;
b—performing ECG verification of good tolerability; and
c—increasing calcium channel blockers dose only if results in step b) are satisfying.

16. The method according to claim 15, wherein the pharmaceutically acceptable salt of dronedarone is hydrochloride.

17. The method according to claim 1, wherein calcium channel blockers are those with depressant effects on the sinus and AV nodes.

18. The method according to claim 6, wherein calcium channel blockers are those with depressant effects on the sinus and AV nodes.

19. The method according to claim 9, wherein calcium channel blockers are those with depressant effects on the sinus and AV nodes.

20. The method according to claim 1, wherein calcium channel blocker is verapamil, diltiazem or bepridil.

21. The method according to claim 6, wherein calcium channel blocker is verapamil, diltiazem or bepridil.

22. The method according to claim 9, wherein calcium channel blocker is verapamil, diltiazem or bepridil.
